(19) United States
(12) Patent Application Publication
(10) Pub. No.: US 2014/0199259 A1
(43) Pub. Date: Jul. 17, 2014

(54) STABILIZED LIQUID FORMULATION

(71) Applicant: Sandoz AG, Basel (CH)

(72) Inventors: Heinrich Franz MATOUS, Kundl (AT); Andreas ZIMMER, Graz (AT)

(21) Appl. No.: 13/903,584

(22) Filed: May 28, 2013

Related U.S. Application Data
(63) Continuation of application No. 13/632,747, filed on Oct. 1, 2012, now abandoned.
Application No. 13/632,747 is a continuation of application No. 13/398,429, filed on Feb. 16, 2012, now abandoned.
Application No. 13/398,429 is a continuation of application No. 13/155,770, filed on Jun. 8, 2011, now abandoned.
Application No. 13/155,770 is a continuation of application No. 12/989,238, now abandoned.

(30) Foreign Application Priority Data
Apr. 26, 2008 (EP) 08008085.6

(51) Int. Cl. A61K 38/27; C07K 16/00; A61K 38/18; A61K 38/19; A61K 38/20; A61K 38/21

(52) U.S. Cl. CPC A61K 38/27; A61K 38/20; A61K 38/21; A61K 38/18; A61K 38/19; C07K 16/00
USPC 424/85.2; 424/85.4; 514/7.6; 514/1.1; 424/130.1; 514/11.4

(57) ABSTRACT

The present invention relates to a liquid formulation comprising an active substance and protamine, wherein the liquid formulation differs from a suspension.

The present invention relates to liquid formulations comprising an active compound and protamine, the use thereof and a process for the preparation of a liquid formulation.

In modern pharmaceutics, liquid formulations represent an important presentation form for pharmaceutically active substances. A particularly relevant sub-group are the so-called parenteral products, which are intended for administration into human or animal tissue by injection or implantation.

Although not limited thereto, parenteral products typically contain those pharmaceutically active compounds which must be supplied directly or rapidly to the patient to be treated such as, for example, pharmaceutically active compounds used in emergency medicine, or also those compounds which can be formulated pharmaceutically only with difficulty by other means. These include, for example, pharmaceutically active compounds based on peptides, polypeptides or proteins.

The preparation of parenteral formulations requires a number of auxiliary substances in order, for example, to ensure isotonicity, to adjust the pH, to improve the solubility, to prevent the degradation of the active substances and in order to achieve adequate antimicrobial properties. These additions should neither influence the medicinal action of the formulation, nor cause local irritation or even be toxic in the concentration used. Such auxiliary substances and additions are known in the prior art.

Peptides, polypeptides and proteins are comparatively new classes of active substance which can predominantly be provided in the required amount and purity with the development of new, efficient synthesis processes or biotechnology processes. Precisely the increasing therapeutic use of polypeptides and proteins, such as, for example, growth factors, cytokines and antibodies, requires measures for providing these active substances in the pharmaceutically active form. This includes, quite generally, stabilizing of the polypeptides and proteins, inter alia prevention of aggregation thereof.

In the field of proteins, the use of human serum albumin, polymeric substances, such as, for example, polyethylene glycol, dextrans or gelatine, urea or individual amino acids has been carried out heuristically to stabilize liquid formulations.

The present invention is based on the object of providing a liquid formulation for a pharmaceutically active substance. The present application is furthermore based on the object of providing a clear liquid formulation for a pharmaceutically active substance. The present invention is moreover based on the object of providing a storage-stable liquid formulation for a pharmaceutically active substance, wherein the pharmaceutically active substance is preferably chosen from the group which comprises peptides, polypeptides and proteins. Finally, the present invention is based on the object of providing process for the preparation of a liquid formulation.

These and further objects are achieved by the subject matter of the present application, and in particular also by the subject matter of the attached independent claims, embodiments emerging from the sub-claims.

According to the invention, the object is achieved in a first aspect by a liquid formulation comprising an active substance and protamine, wherein the liquid formulation differs from a suspension.

In one embodiment, the liquid formulation is a clear liquid formulation.

In a further embodiment, the liquid formulation has an opalescence which is not more intense than the opalescence of reference solution IV as defined in Ph.Eu. 1997, chapter 2.2.1.

In a still further embodiment, the protamine is salmon sperm protamine.

In a still further embodiment, the molar ratio of active substance to protamine is at least 1:2 or more, preferably at least 1:10 and more preferably at least 1:100.

In one embodiment, the molar ratio of active substance to protamine is at least 1:13.

In one embodiment, the active substance is chosen from the group comprising peptides, polypeptides and proteins.

In one embodiment, the active compound has a molecular weight of from about 3,000 Da to about 200,000 Da, preferably about 10,000 to about 100,000 Da.

In one embodiment, the active substance is chosen from the group comprising growth factors, cytokines, interferons, interleukins and antibodies.

In a further embodiment, the active substance is hGH.

In a still further embodiment, the liquid formulation has a pH of from about 4 to 10, more preferably about 5.5 to 7.

In one embodiment, the liquid formulation contains at least one further auxiliary substance, the auxiliary substance being chosen from the group comprising glycine, propylene glycol, mannitol and poloxamer 188.

In one embodiment, the liquid formulation is intended for use in a method for treatment of a disease.

In a second aspect, the object is achieved by the use of protamine for the preparation of a liquid formulation.

In one embodiment of the second aspect, the liquid formulation is a liquid formulation according to the first aspect.

In a third aspect, the object is achieved by the use of protamine as a solubilizing agent.

In one embodiment of the third aspect, protamine is used in the context of the preparation of a liquid formulation, the liquid formulation preferably being a liquid formulation according to the first aspect.

In a fourth aspect, the object is achieved by a process for the preparation of a liquid formulation comprising an active substance and protamine, comprising the steps:

a) provision of protamine;
b) provision of the active substance; and
c) formulation of protamine and the active substance to give a liquid formulation.

In one embodiment of the fourth aspect, the liquid formulation is a liquid formulation according to the first aspect.

The present inventors have found, surprisingly, that liquid formulations of pharmaceutically active compounds, especially if these are those based on peptides, polypeptides or proteins, can be stabilized by addition of protamine, or the pharmaceutically active compound contained in the liquid formulation is stabilized. The present inventors have furthermore found, surprisingly, that the addition of protamine is capable of preventing the crystallization of a pharmaceutically active compound, or at least of reducing this. This applies in particular if the pharmaceutically active compound is a peptide, polypeptide or protein.

The present invention is furthermore based on the finding that protamine can be used as a solubilizing agent in connection with liquid formulations, and in particular clear liquid formulations such as are disclosed herein.

In one embodiment, the term that the formulation is stabilized, as used herein, means that the formulation is stable to storage, preferably the formulation containing protamine has a greater storage stability compared with the formulation without protamine. Preferably, a greater storage stability means that the formulation can be stored over a longer period of time than without the addition of protamine, without one or more changes which limit the storage or storability thereby occurring. Changes which limit the storage or storability are, for example, precipitation or crystallization of the pharmaceutically active compound, opalescence or a discoloration of the liquid stabilizing. These changes can have various causes, depending on the pharmaceutically active agent, such as, for example, complexing, it being possible for the complex to consist of the pharmaceutically active substance or of the pharmaceutically active substance and one or more of the other constituents of the formulation, or degradation of the pharmaceutically active agent.

It is acknowledged by persons skilled in the art that what is said herein for pharmaceutically active compounds applies equally to active substances. To this extent, the terms pharmaceutically active compound and active substance are used synonymously herein, unless explicitly stated otherwise.

The liquid formulation according to the invention is thus a storage-stable liquid formulation which contains at least one active substance and protamine.

The liquid formulation according to the invention containing at least one active substance and protamine differs from a suspension. The term suspension, as used herein, describes in particular a medicament form comprising a flowable, coarsely disperse two-phase system of a liquid external and a solid internal phase, where the system has a continuous external phase in the form of a dispersing agent and flows solely under the influence of gravity, and the particle size of the disperse phase is conventionally 1-100 μm and the content of the solid phase is 0.5-40%, depending on the field of use.

The formulation according to the invention differs in particular from an insulin-containing liquid formulation, which contains insulin and is marketed under the name “Isophan”, and a protamine-containing FSME vaccines, which are in the form of suspensions The formulation according to the invention also differs from a formulation of protamine as an antidote for heparin, this formulation containing no further pharmaceutically active compound in addition to protamine, and in particular no further substance from the pharmaceutically active compound class of peptides, polypeptides or proteins.

In contrast, the liquid formulation according to the invention is a clear liquid formulation. A clear liquid formulation in the context of the present invention is a liquid formulation which has an opalescence which is not more intense than the opalescence of reference solution IV such as is preferably described in the European Pharmacopoeia, also abbreviated herein to Ph.Eu., in particular in chapter 2.2.1 of Ph.Eu., 1997 “Clarity and opalescence of liquids”. The corresponding procedure is also described again herein in Example 1.

Protamine, as preferably used herein, describes a group of polypeptides with a molecular weight of between 1,000 and 6,000, which comprise the amino acid L-arginine to the extent of 80-85%, the remainder of the amino acids which build up these polypeptides being L-alanine, glycine, L-proline, L-serine, L-isoleucine and L-valine. Protamines are compounds with a strongly alkaline reaction. They are obtained from bird or fish sperm by extraction by shaking with dilute acids, the strongly basic protamines forming salts with the acids. Depending on the origin, a distinction is made between clupeine, which is obtained from herring, cyprinine, which is extracted from carp, salmine from salmon and iridine from trout. Protamines are described, for example, in Ando, T., Yamasaki, M., Suzuki, K. “Protamines-Isolation, Characterization Structure and Function”, Springer-Verlag Berlin Heidelberg New York, 1993, pages 1-114. In a preferred embodiment of the liquid formulation according to the invention, the protamine is the free base of salmon sperm protamines, such as is commercially obtainable, for example, from Sigma.

Protamine is contained in the formulation according to the invention or added to this in an amount such that the liquid formulation is clear or remains clear. It lies within the capabilities of the person skilled in the art, in the light of the present disclosure, to determine the absolute amount of protamine as well as the molar ratio of protamine to the active substance by simple routine methods. It is acknowledged by persons skilled in the art that the absolute amount of protamine as well as the molar ratio of protamine to the active substance in the liquid formulation depends on the specific properties of the active substance. Properties of the active substance in this respect are the size and conformation of the active substance and the charge thereof and, associated with this, the isoelectric point thereof.

Consequently, in preferred embodiments the molar ratio of active substance to protamine is at least 1:2 or more. Preferably, the molar ratio is at least 1:10, and still more preferably the molar ratio is at least 1:100.

With respect to the absolute amount of protamine contained in the liquid formulation according to the invention, this is determined in the context of routine investigations on the basis of the above relationship. In this context, starting from the amount of active substance desired or contained in the liquid formulation, the molar ratio and therefore also the absolute amount of protamine such as is to be contained in the concrete liquid formulation is determined.

The pH of the liquid formulation according to the invention is, inter alia also depending on the isoelectric point of the active substance, about 4 to 10, preferably about 6 to 7.

The formulation according to the invention preferably comprises as the active substance one or more active substances which are chosen from the group comprising peptides, polypeptides and proteins. In the context of the present invention also, the active substance differs from a peptide, polypeptide or a protein. In one embodiment, the active substance is a combination of at least two or more active substances. In a preferred embodiment, at least one of the active substances is chosen from the group which comprises peptides, polypeptides and proteins. In the embodiment in which at least one of the active substances is chosen from the group which comprises peptides, polypeptides and proteins, in one alternative the at least one further active substance is likewise chosen from the group which comprises peptides, polypeptides and proteins. In an alternative embodiment to this, the at least one further active compound is not chosen from the group which comprises peptides, polypeptides and proteins. In a still further embodiment, the liquid formulation according to the invention contains two or more, preferably three, four, five, six, seven, eight, nine, ten or more active substances, these active substances preferably being chosen from the group which comprises peptides, polypeptides and proteins.

It is acknowledged that in the embodiments in which the liquid formulation according to the invention contains, in addition to protamine, at least two or more active substances which are chosen individually and independently of one another from the group consisting of peptides, polypeptides and proteins, the protamine content of the liquid formulation must be formed according to the technical teaching given herein. In this case, each active substance is preferably to be regarded individually, i.e. the molar ratio required for each active substance to protamine is to be determined or specified and the amount of protamine resulting from this for each individual active substance is then to be added to the liquid formulation. In this embodiment, the total content of protamine in the liquid formulation according to the invention is then given by the sum of the amount of protamine required or determined for each individual active substance.

In a preferred embodiment, a peptide is defined herein as a polymer comprising several amino acids bonded to one another by amide bonds, the number of amino acids linked to one another preferably not exceeding 50. In the context of the present invention, peptides comprise L-amino acids, D-amino acids or both L- and D-amino acids.

In a preferred embodiment, a polypeptide is understood herein as meaning a peptide, as described above, which comprises more than 50 amino acids, preferably 50 to 100 amino acids.

In a preferred embodiment, a protein is understood herein as meaning a peptide, as described above, which comprises more than 100 amino acids. It is acknowledged that definitions for the terminology other than those given above exist in the prior art.

In a further embodiment, the active substance contained in the liquid formulation according to the invention is one with a molecular weight of from about 3,000 Da to about 200,000 Da, preferably from about 10,000 Da to about 100,000 Da.

In one embodiment, the active substance is chosen from the group comprising growth factors, cytokines and antibodies. Cytokines preferably comprise interleukins, interferons and chemokines. Antibodies preferably comprise polyclonal antibodies and monoclonal antibodies and fragments thereof. Preferred fragments of antibodies are Fab fragments, F(ab)₂ fragments and Fc fragments.

In a preferred embodiment, the active substance is human growth hormone, which is also called hGH or somatropin.

The main fields of indication of somatropin are hypophyseal infantilism in children due to an inadequate secretion of endogenous growth hormone and growth disorders in girls suffering from Ullrich-Turner syndrome, a monosomy X. Nevertheless, treatment is only appropriate if the epiphyseal joints are not yet closed. For successful therapy it is also important that it is started promptly and the dose is continuously adapted to the requirement of the developing child. As a result of the insulin-like action and a positive influence on the cell division rate, somatropin is contraindicated in cases of diabetes mellitus and tumour diseases. It is administered daily via a subcutaneous injection, the best action being achieved by administration in the evening.

Somatropin comprises 191 amino acids bonded to one another by two disulfide bridges (Cys53-Cys1165, Cys182-Cys189). In solution, somatropin has similarity with a globular protein, but the three-dimensional structure contains 4 ct-helices bonded to one another by lengthened extended chains. The molecule contains 3 methionines, 2 of which (Met14, Met125) are sensitive to oxidation. The methionine in position 170 is probably shielded from oxidation processes due to its position inside the molecule. A somatropin molecule moreover contains 9 asparagines and 13 glutamines and therefore many reactive starting points for a deamination reaction. Asn 149 is stated as the main point of attack for the deamination.

The deamination of somatropin takes place via the intermediate stage of a cyclic imide, which rearranges into an iso-aspartic acid by hydrolysis. The tendency to form the cyclic imide depends on the flexibility of the adjacent C-terminal amino acid. This also explains why Asn 149 has a higher reactivity than Asn 152. This isomerization can be detected by the protein isoaspartyl methyltransferase reaction. Asn 130 is also a possible isomerization site in the molecule. Like many other globular proteins, somatropin also tends to form aggregates, which can be formed by either covalent or non-covalent bonds. Both shaking processes and freezing of the aqueous solution of somatropin can contribute towards the formation of soluble and insoluble aggregates. It is these undesirable reactions which are suppressed or at least slowed down by means of the liquid formulation according to the invention.

In the embodiment of the liquid formulation according to the invention in which the active substance is somatropin, the molar ratio of somatropin to protamine is at least 1:13. The absolute amounts of somatropin and protamine correspond to those such as are mentioned in the formulations described in the examples part. Preferably, the pH of this liquid formulation is between 5.5 and 7, preferably between 5.8 and 6.5.

In one embodiment, the liquid formulation according to the invention is an aqueous liquid formulation which, in addition to the active substance and protamine, also contains further constituents which are conventional for liquid formulations and in particular parenteral products and are known to persons skilled in the art in the field. The conventional constituents include, for example, buffers, agents for adjusting the tonicity, solubilizing agents, preservatives etc.

In one embodiment, the buffer is a phosphate buffer based on sodium dihydrogen phosphate and disodium hydrogen phosphate.

1,2-Propylene glycol can be used as a preservative, to adjust the tonicity or also as a solubilizing agent. Depending on the intended use, the content in the liquid formulation varies and is, for example, 2.3 wt. % when it is used as an agent for adjusting the isotonicity. Preferably, the compound is a clear, colourless liquid, which is viscous and has a low volatility. The substance is odourless and hygroscopic, has a boiling point of 184-189° C. and is miscible with water, lower alcohols, esters and ketones in any ratio.

In one embodiment, the liquid formulation according to the invention contains a surface-active solubilizing agent, the surface active solubilizing agent being poloxamer 188. Poloxamers are polyols of a series of closely related block copolymers of ethylene oxide and propylene oxide. In the case of poloxamer 188, the average molecular weight is 7,680 to 9,510, the content in the polymer of ethylene oxide contents (a) being 2×80 and the content of propylene oxide groups (b) being 27.

Mannitol is an example of a physiologically inert auxiliary substance, which acts as a structure-forming agent, but also as a stabilizer, the stabilizing certainly taking place via the OH groups. The content of mannitol in pharmaceutical formulations can be up to 50 wt. %.

Glycine is used to adjust the pH, and also the tonicity. Typical concentrations of glycine in pharmaceutical formulations are up to 90 wt. %. In a preferred embodiment, the liquid formulation contains glycine.

In the field of liquid formulations, so-called auxiliary substance solutions are used which form the base for the liquid formulation and to which in particular the active substance and optionally further constituents of the liquid formulation are added. Examples of auxiliary substance solutions are the mannitol formulation described in Example 2, and the glycine formulation and glycine/propylene glycol formulation. In the context of the present invention, these auxiliary substance solutions can also be used in connection with active substances other than the somatropin mentioned in the examples.

In one embodiment of the liquid formulation according to the invention, this is a pharmaceutical liquid formulation.

In a further embodiment, the liquid formulation according to the invention is used in the treatment of a disease or for the preparation of a medicament for treatment of a disease. It is acknowledged by persons skilled in the art that the disease which can be treated using the liquid formulation according to the invention is determined by the active substance contained in the liquid formulation. The term treatment as used herein also includes the prevention of the corresponding disease.

Finally, the present invention relates to a process for the preparation of a liquid formulation comprising an active substance and protamine, comprising the steps:

a) provision of protamine;
b) provision of the active substance or the active substances; and
c) formulation of protamine and the active substance to give a liquid formulation.

In principle, the process can be carried out by conventional procedures and methods known in the field to persons skilled in the art. In one embodiment, the protamine is provided in an auxiliary substance solution, in particular one such as is described herein, and the active substance is then added. However, in the context of the present invention, the active substance is first added to the auxiliary substance solution and the protamine is then added. In one embodiment, the pH of the auxiliary substance solution is adjusted such that on addition of protamine or the active substance both the isoelectric point of protamine and that of the active substance are avoided, or passing through the isoelectric point(s) is avoided. Finally, in one embodiment glycine is added to the liquid formulation, preferably in an auxiliary substance solution.

### EXAMPLE 1

### Method for Determination of the Opalescence of Liquid Formulations

The method described below for determination of the opalescence of liquid formulations corresponds to the method described in Ph.Eu. 1997, chapter 2.2.1.

In identical Nessler cylinders of colourless, transparent neutral glass with an internal diameter of from 15 to 25 mm and with a flat base, the liquid to be tested is compared with the freshly prepared reference suspension described below in a layer thickness of 40 mm. 5 min after preparation of the reference suspension the liquids are examined by inspecting vertically against a dark background and in diffuse daylight.

The distribution of light must be such that the reference suspension I is easy to distinguish from water R and the reference suspension II is easy to distinguish from reference suspension I.

A liquid is described as clear if the clarity under the conditions given above corresponds to that of water R or of the solvent used or if the liquid is not more opalescent than reference suspension I.

### Reagents

Hydrazine sulfate solution: 1.0 g of hydrazine sulfate R is dissolved in water R to 100.0 ml. The solution is left to stand for 4 to 6 h.

Methenamine solution: 2.5 g of methenamine R are dissolved in 25.0 ml of water R in a 100 ml conical flask with a ground glass stopper.

Opalescence stock suspension: 25.0 ml of the hydrazine sulfate solution are introduced into the conical flask with the methanamine solution, the components are mixed and the mixture is left to stand for 24 h. This suspension can be stored for 2 months in a glass container with an intact surface. The suspension should not adhere to the wall of the container and must be shaken thoroughly before use.

Opalescence reference suspension: 15.0 ml of opalescence stock suspension are diluted with water R to 1,000.0 ml. The suspension is to be freshly prepared as required and should be used for at most 24 h.

Reference suspension: The reference suspensions are prepared according to the following table. They are prepared and shaken immediately before use.

| | I | II | III | IV |
|---|---|---|---|---|
| Opalescence | 5.0 ml | 10.0 ml | 30.0 ml | 50.0 ml |
| reference suspension: | | | | |
| Water R | 95.0 ml | 90.0 ml | 70.0 ml | 50.0 ml |

### EXAMPLE 2

### Auxiliary Substance Formulations

The following auxiliary substance formulations can be used in the context of the present invention.

Recipe of the somatropin-containing liquid formulation based on the mannitol-containing auxiliary substance formulation. The auxiliary substance formulation corresponds to the following recipe without the active substance somatropin.

| Constituents | Molecular weight [g/mol] | mg/ml of formulation |
|---|---|---|
| Active substance | | |
| Somatropin | 22,129 | 3.33 mg/ml (151 μM) |
| Auxiliary substances | | |
| Na₂HPO₄ × 7H₂O | 268.07 | 0.88 mg/ml (3.2 mM) |
| NaH₂PO₄ × 2H₂O | 156.01 | 1.039 mg/ml (6.7 mM) |
| Mannitol | 182.18 | 35.11 mg/ml (192.7 mM) |
| Poloxamer 188 | 8,595 | 1.98 mg/ml (231 μM) |
| Benzyl alcohol | 108.14 | 8.93 mg/ml (82.6 mM) |
| Water | 18.02 | to 12.26 ml |
| H₃PO₄ | 98.00 | to pH = 6.2 |
| Protamine | 4,100 | 82 mg/ml-0.001 μg/ml |

Recipe of the somatropin-containing liquid formulation based on the glycine-containing auxiliary substance formulation. The auxiliary substance formulation corresponds to the following recipe without the active substance somatropin.

| Constituents | Molecular weight [g/mol] | mg/ml of formulation |
|---|---|---|
| Active substance | | |
| Somatropin | 22,129 | 3.33 mg/ml (151 μM) |
| Auxiliary substances | | |
| Na₂HPO₄ × 7H₂O | 268.07 | 0.88 mg/ml (3.2 mM) |
| NaH₂PO₄ × 2H₂O | 156.01 | 1.039 mg/ml (6.7 mM) |
| Glycine | 75.07 | 7.43 mg/ml (98.9 mM) |
| Poloxamer 188 | 8,595 | 1.98 mg/ml (231 μM) |
| Benzyl alcohol | 108.14 | 8.93 mg/ml (82.6 mM) |
| Water | 18.02 | to 12.26 ml |
| H₃PO₄ | 98.00 | to pH = 6.2 |
| Protamine | 4,100 | 75 mg/ml-0.001 μg/ml |

Recipe of the somatropin-containing liquid formulation based on the glycine/propylene glycol-containing auxiliary substance formulation. The auxiliary substance formulation corresponds to the following recipe without the active substance somatropin.

| Constituents | Molecular weight [g/mol] | mg/ml of formulation |
|---|---|---|
| Active substance | | |
| Somatropin | 22,129 | 3.33 mg/ml (151 μM) |
| Auxiliary substances | | |
| Na₂HPO₄ × 7H₂O | 268.07 | 0.88 mg/ml (3.2 mM) |
| NaH₂PO₄ × 2H₂O | 156.01 | 1.039 mg/ml (6.7 mM) |
| Glycine | 75.07 | 7.43 mg/ml (98.9 mM) |
| Propylene glycol | 76.10 | 7.43 mg/ml (97.6 mM) |
| Poloxamer 188 | 8,595 | 1.98 mg/ml (231 μM) |
| Benzyl alcohol | 108.14 | 8.93 mg/ml (82.6 mM) |
| Water | 18.02 | to 12.26 ml |
| H₃PO₄ | 98.00 | to pH = 6.2 |
| Protamine | 4,100 | 82 mg/ml-0.02 μg/ml |

### EXAMPLE 3

### Preparation of Various Protamine-Containing Liquid Formulations

In the preparation of the various liquid formulations, a stock solution containing somatropin, also called the bulk solution, was used as the starting material. The bulk solution contains the somatropin, prepared by a recombinant method, to the extent of 10.1 mg/ml, as determined by size exclusion chromatography.

The batches were prepared on a small scale. Since it was necessary to thaw the bulk solution gently from −20° C. to room temperature before each experiment, the particular size of the experiment was matched to the portions of active substance solution available in order to prevent renewed freezing and thawing of the sensitive protein.

In order to ensure sterility during the preparation process, the formulations were prepared aseptically under a laminar flow box. Furthermore, it was ensured that each of the components was filtered through a syringe tip filter (Acrodisc Syringe Filter, Gelman Laboratory) with a defined pore diameter of 0.22 μm before use.

In the following and in Example 4, the various protamine-containing liquid formulations are also called “experiments”. The basis for at least some of the experiments was the formulation called EP200AQ formulation, which has the following composition:

| Constituents | Molecular weight [g/mol] | mg/ml of formulation |
|---|---|---|
| Active substance | | |
| Somatropin | 22,129 | 3.33 mg/ml (151 μM) |
| Auxiliary substances | | |
| Na₂HPO₄ × 7H₂O | 268.07 | 0.88 mg/ml (3.2 mM) |
| NaH₂PO₄ × 2H₂O | 156.01 | 1.039 mg/ml (6.7 mM) |
| Mannitol | 182.18 | 35.11 mg/ml (192.7 mM) |
| Poloxamer 188 | 8,595 | 1.98 mg/ml (231 μM) |
| Benzyl alcohol | 108.14 | 8.93 mg/ml (82.6 mM) |
| Water | 18.02 | to 12.26 ml |
| H₃PO₄ | 98.00 | to pH = 6.2 |

Preparation process of Experiments 1 and 2
2,090 μl of bulk solution (pH 7.3) are initially introduced into the mixing vessel
910 μl of aqueous protamine solution (0.84 μg of protamine/ml of formulation) are slowly added, which corresponds to 90% of the water of the entire experiment (strongly basic, pH increases with increasing protamine concentration)
3,040 μl of auxiliary substance solution are then metered in (pH 3.80)
in the solution obtained in this way a pH of 6.2 is established with 1 M H₃PO₄
topping up with aqua bidist. to the required final volume

### Experiment 1

In this experiment, an EP2000AQ formulation free from active substance was prepared. The aqueous protamine solution, which contains 90% of the total water of the formulation, was added to the buffer solution, which is this case comprises only phosphates and aqua bidist., the auxiliary substance solution was then metered in, a pH of 6.2 was established with 1 M H₃PO₄ and the mixture was topped up with water to the required final weight.

### Experiment 2

In this experiment, an EP2000AQ formulation with a molar ratio of hGH to protamine of 6:1 (0.84 mg/ml of formulation) was formed. The components were combined analogously to Experiment 1.

Preparation process of batches 3, 4, 5, 6, 7
2,090 μl of bulk solution (pH 7.3) are initially introduced into the mixing vessel
505 μl of water are added, which corresponds to 50% of the water of the entire experiment
3,040 μl of auxiliary substance solution are then metered in (pH 3.80)
at this point, 404 μl of the aqueous protamine solution are added
in the solution obtained in this way, a pH of 6.2 is established with 1 M H₃PO₄
topping up with aqua bidist. to a final volume of 6,130 μl

### Experiment 3

In the course of this experiment, an EP2000AQ formulation with molar hGH/protamine ratios of 1:0.2 (0.4 μg of protamine/ml of formulation) and 1:0.002 (0.04 μg of protamine/ml of formulation) was prepared. The preparation process was modified inasmuch as the protamine solution was not metered directly into the bulk solution, but a type of “buffer zone” was created between the two proteins by the addition of water and auxiliary substance solution.

### Experiment 4

In this experiment also, the protamine solution was metered in after addition of the auxiliary substance solution. The molar ratio between the two proteins is hGH to protamine 1:0.04 (0.08 μg of protamine/ml of formulation).

### Experiment 5

The preparation of the formulation is carried out in accordance with batches 3 and 4. The molar ratio between hGH and protamine was modified from 1:0.04 to 1:0.1 (2 μg of protamine/ml of formulation) or 1:0.2 (4 μg of protamine/ml of formulation).

### Experiment 6

In this experiment the preparation process was retained. The molar ratios range from 1:0.1 (2 μg of protamine/ml of formulation) via 1:0.08 (1.6 μg of protamine/ml of formulation), 1:0.06 (1.2 μg of protamine/ml of formulation), 1:0.04 (0.8 μg of protamine/ml of formulation) to 1:0.02 (0.4 μg of protamine/ml of formulation). The aqueous protamine solutions resulted by dilution of a protamine stock solution with aqua bidist.

### Experiment 7

In the course of this experiment, the mannitol auxiliary substance solutions was replaced by a glycine/propylene glycol auxiliary substance solution (for the recipe, see Table 10). The protamine solutions were again prepared by dilution of a protamine stock solution with aqua bidist. As in Experiment 6, in this case also formulations with molar ratios of hGH to protamine of from 1:0.1 (2 μg of protamine/ml of formulation) to 1:0.02 (0.4 μg of protamine/ml of formulation) were prepared.

### Experiments 8, 9, 10

In accordance with the formulation work to date, the next consideration was to what extent each individual auxiliary substance has an influence on possible interactions between hGH and protamine. For this purpose, mannitol and glycine/propylene glycol formulations were prepared in parallel, in each case one component of the auxiliary substance solution being removed per experiment. Starting from benzyl alcohol via poloxamer 188, propylene glycol and mannitol, the number of components was reduced continuously, so that in the end an experiment with buffer solution and glycine and one which consisted only of buffer solution were available. Immediately after the preparation, the final formulations were measured by means of a pH meter.

### Experiment 11

In order to ensure the reproducibility of the results, a further series was prepared with mannitol auxiliary substance solution or glycine/propylene glycol auxiliary substance solution. Nothing was changed in the preparation process, and the molar ratios of hGH and protamine were as follows: 1:0.2 (4 μg of protamine/ml of formulation); 1:0.1 (2 μg of protamine/ml of formulation); 1:0.08 (1.6 μg of protamine/ml of formulation); 1:0.06 (1.2 μg of protamine/ml of formulation), 1:0.04 (0.8 μg of protamine/ml of formulation) and 1:0.02 (0.4 μg of protamine/ml of formulation).

In order to be certain during the sterile filtration that the filter is saturated with protamine, a larger batch was calculated for the protamine stock solution and the filter was flushed beforehand with approx. 3 ml of protamine solution. In this case also, the pH of the final formulations was determined directly after the preparation.

Preparation process of Experiments 12, 13, 14, 15
2,090 μl of bulk solution (pH 7.3) are initially introduced into the mixing vessel
1,010 μl of water are added, which corresponds to 50% of the water of the entire experiment
6,080 μl of auxiliary substance solution are then metered in (pH 3.80)
at this point, 808 μl of the aqueous protamine solution are added
in the solution obtained in this way, a pH of 6.2 is established with 1 M H₃PO₄ or 0.1 M H₃PO₄
topping up with aqua bidist. to a final volume of 6,130 μl

### Experiment 12

On the basis of the results so far, it was decided to ignore the mannitol formulation for the time being, and the glycine/propylene glycol and the glycine auxiliary substance solution were concentrated on. It was furthermore decided to double the experiment, which also facilitated the preparation of the protamine stock solution.

hGH and protamine were employed in molar ratios of 1:0.5 (10 μg of protamine/ml of formulation); 1:0.2 (4 μg of protamine/ml of formulation); 1:0.1 (2 μg of protamine/ml of formulation); 1:0.05 (1 μg of protamine/ml of formulation), 1:0.02 (0.4 μg of protamine/ml of formulation) and 1:0.01 (0.2 μg of protamine/ml of formulation.

In addition, in order to facilitate the pH correction, a 0.1 M H₃PO₄ was used alternatively to a 1 M H₃PO₄. In this experiment also, after the preparation the final formulations were measured in a pH meter and then corrected to a value of 5.8. In order to be able to store the samples for stability tests, a pH of 6.2 was finally established with 0.5 M sodium hydroxide solution.

### Experiment 13

Two different auxiliary substance solutions were employed. Auxiliary substance solution I contains glycine and propylene glycol, auxiliary substance solution II contains mannitol.

This experiment differs from the previous inasmuch as the pH of the formulation was adjusted to a value of 7 with 0.5 N sodium hydroxide solution after addition of the bulk solution, water and auxiliary substance solution, the pH of this mixture of bulk solution, water and auxiliary substance solution first having been determined per se. The molar ratios used were as follows: hGH to protamine 1:0.05 (1 μg of protamine/ml of formulation); 1:0.02 (0.4 μg of protamine/ml of formulation) and 1:0.01 (0.2 μg of protamine/ml of formulation).

After correction to a pH of 7 and metering in of the aqueous protamine solution and the remaining water, the pH of the final formulation was determined, and the extent to which it can be lowered before hGH reaches its IP and precipitates out was tested.

### Experiment 14

The mannitol auxiliary substance solution was used. The hGH/protamine ratio was again 1:0.05 (1 μg of protamine/ml of formulation); 1:0.02 (0.4 μg of protamine/ml of formulation) and 1:0.01 (0.2 μg of protamine/ml of formulation).

In contrast to Experiment 13, the pH of the solutions was determined here only after the formulations had been prepared. Thereafter, the extent to which the pH can be lowered before precipitation of hGH occurs was again tested.

### Experiment 15

In contrast to the preceding experiments, in this test series the protamine concentration was lowered both in the mannitol and in the glycine/propylene glycol experiment. The formulations contain hGH and protamine in molar ratios of 1:0.005 (0.1 μg of protamine/ml of formulation) and 1:0.001 (0.02 μg of protamine/ml of formulation). The pH of the formulation was adjusted to a value of 7 with 0.5 N sodium hydroxide solution after mixing of bulk solution, water and auxiliary substance solution. The protamine solution was then added, the mixture was topped up with water to a target volume of 12,260 μl and the pH was adjusted to a value of approximately 5.

Preparation process of Experiments 16, 17, 18, 19
correction of the pH of the auxiliary substance solution to a required value
introduction of protamine as an additional component of the auxiliary substance solutions; protamine is dissolved directly in the auxiliary substance solution
dilution of the stock auxiliary substance solution with further auxiliary substance solution in order to achieve the desired protamine concentration
cautious metering in of the bulk solution
adjustment of the target pH of the final formulation
centrifuging off of any precipitates which occur
transfer into 2R vials and storage at room temperature or at 2-8° C.

### Experiment 16

In this experiment, the protamine was dissolved in the auxiliary substance solutions both for the mannitol and for the glycine/propylene glycol experiment, the bulk solution was then metered in and the pH was determined Before dissolving of the protamine in the auxiliary substance solution, a pH of 7 was established in this. 330 μl of the bulk solution were added in 30 μl steps with constant shaking. The molar ratio of hGH to protamine is 1:267 (75 μg of protamine/ml of formulation). Thereafter, the pH was lowered to a target pH of 6.00 with 1 M H₃PO₄.

### Experiment 17

This experiment differs from Experiment 16 in that the auxiliary substance solutions in this case have a pH of 3.6 instead of 7.0. The molar ratio of hGH to protamine is again 1:267 (75 μg of protamine/ml of formulation).

In this case also, the pH was corrected to a target pH of 6.00.

### Experiments 18, 19

Since the modification to the preparation process had proved itself, the size of the experiments was increased considerably in order to be able to store sufficient examples for stability tests for subsequent analysis. Both the mannitol and the glycine auxiliary substance solution were employed. After dissolving the protamine in the auxiliary substance solutions, a pH of 7 was in each case established. In each case four hGH/protamine molar ratios were prepared and these were corrected to four different pH values. The molar ratios used were hGH to protamine 1:100 (53.5 μg of protamine/ml of formulation); 1:10 (5.35 μg of protamine/ml of formulation) and 1:1 (0.53 μg of protamine/ml of formulation) and 1:0.1 (0.05 μg of protamine/ml of formulation).

The pH established with 1 M H₃PO₄, 0.1 M H₃PO₄ and 0.1 M sodium hydroxide solution ranged from 5.8 via 6.0 and 6.2 to a value of 6.5.

### EXAMPLE 4

### Stability of the Protamine-Containing Liquid Formulations

The results on the stability of the protamine-containing liquid formulations described in Example 3 and in particular of the influence of protamine on the stability, measured on the basis of the opalescence of the liquid formulation, are summarized in this example.

The above results with respect to the stability of somatropin in protamine-containing liquid formulations were also confirmed by CD spectroscopy.

### Experiment 1

After addition of all the components, the formulation which contained no active substance had a pH of 10 and could be corrected to a value of 6.2 with 1 M phosphoric acid without problems.

| Exper- | Molar ratio | | | | | | |
|---|---|---|---|---|---|---|---|
| iment | hGH:protamine | Comp. 1 | Comp. 2 | Comp. 3 | Comp. 4 | Comp. 5 | Comp. 6 |
| 1 | | | aqueous | auxiliary | pH | aqua bidist. | |
| | | | protamine | substance | adjustment | | |
| | | | solution | solution | to 6.2 | | |
| | | | | (mannitol) | | | |
| 2 | 6:1 | bulk | aqueous | auxiliary | pH | aqua bidist. | |
| | | solution | protamine | substance | adjustment | | |
| | | | solution | solution | to 6.2 | | |
| | | | | (mannitol) | | | |
| 3 | 1:0.02 | bulk | aqua bidist. | auxiliary | aqueous | pH adjustment | top up with |
| | 1:0.002 | solution | (50% of | substance | protamine | to 6.2 | aqua bidist. |
| | | | the total | solution | solution | | to final |
| | | | water) | (mannitol) | | | weight |
| 4 | 1:0.04 | bulk | aqua bidist. | auxiliary | aqueous | pH adjustment | top up with |
| | | solution | (50% of | substance | protamine | to 6.2 | aqua bidist. |
| | | | the total | solution | solution | | to final |
| | | | water) | (mannitol) | | | weight |
| 5 | 1:0.1; 1:0.2 | bulk | aqua bidist. | auxiliary | aqueous | pH adjustment | top up with |
| | | solution | (50% of | substance | protamine | to 6.2 | aqua bidist. |
| | | | the total | solution | solution | | to final |
| | | | water) | (mannitol) | | | weight |
| 6 | 1:0.1. 1:0.08 | bulk | aqua bidist. | auxiliary | aqueous | pH adjustment | top up with |
| | 1:0.06. 1:0.04. | solution | (50% of | substance | protamine | to 6.2 | aqua bidist. |
| | 1:0.02 | | the total | solution | solution | | to final |
| | | | water) | (mannitol) | | | weight |
| 7 | 1:0.1. 1:0.08 | bulk | aqua bidist. | auxiliary | aqueous | pH adjustment | top up with |
| | 1:0.06. | solution | (50% of | substance | protamine | to 6.2 | aqua bidist. |
| | 1:0.041:0.02 | | the total | solution (glycine/ | solution | | to final |
| | | | water) | propylene | | | weight |
| | | | | glycol) | | | |
| 8. 9. | 1:0.1.1:0.08 | bulk | aqua bidist. | auxiliary | aqueous | pH adjustment | top up with |
| 10. 11 | 1:0.06.1:0.04 | solution | (50% of | substance | protamine | to 6.2 | aqua bidist. |
| | 1:0.02 | | the total | solution | solution | | to final |
| | | | water) | (mannitol | | | weight |
| | | | | and glycine/ | | | |
| | | | | propylene | | | |
| | | | | glycol) | | | |
| 12 | 1:0.5; 1:0.2; | bulk | aqua bidist. | auxiliary | aqueous | pH adjustment | top up with |
| | 1:0.1 | solution | (50% of | substance | protamine | to 6.2 | aqua bidist. |
| | 1:0.05; 1:0.02 | | the total | solution (glycine | solution | | to final |
| | 1:0.01 | | water) | and | | | weight |
| | | | | glycine/ | | | |
| | | | | propylene | | | |
| | | | | glycol) | | | |
| 13 | 1:0.05 1:0.02 | bulk | aqua bidist. | auxiliary | pH | aqueous protamine | top up with |
| | 1:0.01 | solution | (50% of | substance | adjustment | solution | aqua bidist. |
| | | | the total | solution | to 7 | | to final |
| | | | water) | (mannitol | | | weight |
| | | | | and glycine/ | | | |
| | | | | propylene | | | |
| | | | | glycol) | | | |
| 14 | 1:0.05; 1:0.02 | bulk | aqua bidist. | auxiliary | aqueous | top up with | determination |
| | 1:0.01 | solution | (50% of | substance | protamine | aqua bidist. to | of the final |
| | | | the total | solution | solution | final weight | pH |
| | | | water) | (mannitol) | | | |
| 15 | 1:0.005; 1:0.001 | bulk | aqua bidist. | auxiliary | pH | aqueous protamine | top up with |
| | | solution | (50% of | substance | adjustment | solution | aqua bidist. |
| | | | the total | solution | to 7 | | to final |
| | | | water) | (mannitol) | | | weight |
| 16. 17 | 1:267 | dissolve | bulk | determination | lowering of | | |
| | | protamine in | solution | of the | the pH to | | |
| | | mannitol and glycine/ | | pH | a final pH | | |
| | | propylene glycol | | | of 6 | | |
| | | auxiliary substance | | | | | |
| | | solution | | | | | |
| 18. 19 | 1:100; 1:10; 1:1 | dissolve | adjustment | bulk | determination | correction of the | |
| | 1:0.1 | protamine in | of the pH | solution | of the final | pH of the | |
| | | mannitol and glycine | of the | | pH | final formulations | |
| | | auxiliary substance | auxiliary | | | to 5.8; 6.0; 6.2; 6.5 | |
| | | solution (pH = 3.6) | substance | | | | |
| | | | solutions to | | | | |
| | | | a value of 7 | | | | |

Molar ratios of hGH:protamine of Experiments 1 to 19

### Experiment 2

This is the case of an EP2000AQ formulation containing active substance. After adding aqueous protamine solution, a white flocculent precipitate had formed, which had settled on the bottom after approximately five minutes.

Preparation process of experiments 3, 4, 5, 6, 7
2,090 μl of bulk solution (pH 7.3) are initially introduced into the mixing vessel
505 μl of water are added, which corresponds to 50% of the water of the entire experiment
3,040 μl of auxiliary substance solution are then metered in (pH 3.80)
at this point, 404 μl of the aqueous protamine solution are added
in the solution obtained, a pH of 6.2 is established with 1 M H₃PO₄
topping up with aqua bidist. to a final volume of 6,130 μl

### Experiment 3

Due to the precipitate which had formed immediately after the addition of the protamine solution to the bulk solution during Experiment 2, the preparation process for the formulation in this experiment was modified inasmuch as the protamine solution was added only after addition of the auxiliary substance solution.

Although a brief opalescence was to be observed at the point of dropwise addition of the protamine solution, which is probably to be attributed to a concentration-related interfacial phenomenon, both the hGH to protamine of 1:0.02 (0.4 μl of protamine/ml of formulation) and the 1:0.002 formulation (0.04 μl of protamine/ml of formulation) remained clear after slow stirring. Neither further opalescence nor a precipitate occurred.

### Experiment 4

After metering in all the components, the batch with molar ratios of hGH to protamine of 1:0.04 (0.8 μg of protamine/ml of formulation) remained clear, and no visible precipitate formed.

### Experiment 5

In these formulations, at both the hGH/protamine ratios opalescence already occurred at the point of dropwise addition during the preparation of the protamine solution, and could not be eliminated again by slow stirring. After a few minutes, clearly visible aggregates were to be observed.

### Experiment 6

In this experiment also, the preparation process of earlier experiments was adopted and each of the components employed was subjected to sterile filtration before the metering in. At all the dilutions a slight opalescence was to be observed, the hGH/protamine batch with a molar ratio of 1:0.1 (2 μg of protamine/ml of formulation) being more intensely opalescent than that which has a molar ratio of 1:0.02 (0.4 μg of protamine/ml of formulation).

### Experiment 7

In this experiment, as in experiment 6, the sugar alcohol mannitol was replaced by the stabilizers glycine and propylene glycol. The molar ratios between hGH and protamine ranged from 1:0.1 (2 μg of protamine/ml of formulation) via 1:0.08 (1.6 μg of protamine/ml of formulation), 1:0.06 (1.2 μg of protamine/ml of formulation), 1:0.04 (0.8 μg of protamine/ml of formulation) to 1:0.02 (0.4 μg of protamine/ml of formulation). At all the dilutions streakiness occurred after addition of the auxiliary substance, which dissolved again after slow shaking. Neither the 1:0.1 nor the 1:0.08 dilution showed opalescence after addition of the protamine solution. From this result it was possible to deduce that glycine, as an amphiphilic molecule, probably interacts with both proteins and therefore can prevent aggregate formation. The final formulations of this experiments was all dissolved to clear solutions.

Preparation process of Experiments 8, 9, 10, 11

In accordance with the formulation work so far, the next consideration was to what extent each individual auxiliary substance has an influence on possible interactions between hGH and protamine. For this purpose, mannitol and glycine/propylene glycol formulations were prepared in parallel, in each case one component of the auxiliary substance solution being removed per experiment.

Starting from benzyl alcohol via poloxamer 188, propylene glycol and mannitol, the number of components was reduced continuously. Experiment 11 consisted of buffer solution and glycine or the pure buffer solution Immediately after the preparation, the final formulations were measured by means of a pH meter.

### Experiments 8, 9, 10

In these experiments, which contain on the one hand mannitol and on the other hand glycine and propylene glycol, molar ratios of hGH to protamine in the range of 1:0.1 via 1:0.08, 1:0.06, 1:0.04 to 1:0.02 were again prepared.

### Experiment 8

All the components of the formulations were present: After addition of the auxiliary substance solution, a transient streakiness was to be observed at each dilution. The IP of hGH was probably passed through. Both at a molar ratio of 1:0.1 and at that of 1:0.08, no opalescence was to be observed in the final formulation. However, it must be reckoned that due to the small batch quantity, some of the protamine was adsorbed on the sterile filter and therefore the desired protamine concentration was not present in the final formulation. In later experiments the amount of protamine solution was therefore increased and the sterile filter was saturated with protamine beforehand.

### Experiment 9

In these two formulations, the addition of the preservative benzyl alcohol was in each case omitted during the preparation. The molar ratios were retained, opalescence of the final formulation again being observed neither at a molar ratio of 1:0.1 nor at one of 1:0.08. In this case also, it must be reckoned that the experiment does not contain the desired protamine concentration.

The following pH values were to be determined for the final formulation:

| | | | | pH |
|---|---|---|---|---|
| | Appearance | pH | Appearance | Gly/PG |
| Molar ratio | mannitol | mannitol | Gly/PG | formu- |
| hGH:protamine | formulation | formulation | formulation | lation |
| 1:0.1 | clear solution | 6.14 | clear solution | 4.16 |
| 1:0.08 | clear solution | 5.82 | clear solution | 4.10 |
| 1:0.06 | clear solution | 6.33 | clear solution | 4.25 |
| 1:0.04 | clear solution | 4.88 | clear solution | 4.14 |
| 1:0.02 | clear solution | 6.0 | clear solution | 4.12 |

### Experiment 9

### PH of the Final Formulations

### Experiment 10

The recipes used in this experiment contain neither benzyl alcohol nor propylene glycol. Here also a brief streakiness was to be observed. The final formulations are all dissolved to clear solutions and the protamine concentration is probably lower than intended due to adsorption phenomena.

The following table shows the pH of the final formulations:

| | | | | pH |
|---|---|---|---|---|
| | Appearance | pH | Appearance | Gly/PG |
| Molar ratio | mannitol | mannitol | Gly/PG | formu- |
| hGH:protamine | formulation | formulation | formulation | lation |
| 1:0.1 | clear solution | 6.02 | clear solution | 4.27 |
| 1:0.08 | clear solution | 6.12 | clear solution | 4.07 |
| 1:0.06 | clear solution | 6.23 | clear solution | 4.15 |
| 1:0.04 | clear solution | 6.33 | clear solution | 4.11 |
| 1:0.02 | clear solution | 6.23 | clear solution | 4.16 |

### Experiment 10

### PH of the Final Formulations

### Experiment 11

In order to saturate the filter with protamine before the sterile filtration, a larger batch was calculated for the protamine stock solution. In the solutions containing glycine and propylene glycol, a brief opalescence, which disappeared again, was to be observed after addition of the auxiliary substance solution. The final formulations of the glycine/propylene glycol batch are all dissolved to clear solutions, and in the mannitol batch an opalescence was to be seen from a molar ratio of hGH to protamine of 1:0.06 (1.2 μg of protamine/ml of formulation) with increasing protamine concentration.

It is assumed that in this experiment the samples contain the desired amounts of protamine and as a result opalescence of the mannitol-containing solutions again occurred from a certain protamine concentration.

Measurement of the final formulations in a pH meter gave the following result:

| | | | | pH |
|---|---|---|---|---|
| | Appearance | pH | Appearance | Gly/PG |
| Molar ratio | mannitol | mannitol | Gly/PG | formu- |
| hGH:protamine | formulation | formulation | formulation | lation |
| 1:0.1 | opalescence | 6.32 | clear solution | 4.26 |
| 1:0.08 | opalescence | 6.45 | clear solution | 4.28 |
| 1:0.06 | slightly | 6.44 | clear solution | 4.27 |
| | opalescent | | | |
| 1:0.04 | dissolved to a | 6.36 | clear solution | 4.24 |
| | clear solution | | | |
| 1:0.02 | dissolved to a | 6.34 | clear solution | 4.32 |
| | clear solution | | | |

### Experiment 11

### PH of the Formulations

Preparation process of Experiments 12, 13, 14, 15
2,090 μl of bulk solution (pH 7.3) are initially introduced into the mixing vessel
1,010 μl of water are added, which corresponds to 50% of the water of the entire experiment
6,080 μl of auxiliary substance solution are then metered in (pH 3.80)
at this point, 808 μl of the aqueous protamine solution are added
in the solution obtained in this way, a pH of 6.2 is established with 1 M H₃PO₄ or 0.1 M H₃PO₄
topping up with aqua bidist. to a final volume of 6,130 μl

### Experiment 12

As already mentioned, from this point in time attention was chiefly paid to the glycine/propylene glycol or glycine formulation, since mannitol-containing solutions rather tended towards opalescent solutions. hGH and protamine were employed in the molar ratios of 1:0.5 (0.10 μg of protamine/ml of formulation); 1:0.2 (0.04 μg of protamine/ml of formulation); 1:0.1 (0.02 μg of protamine/ml of formulation); 1:0.05 (0.01 μg of protamine/ml of formulation; 1:0.02 (0.004 μg of protamine/ml of formulation) and 1:0.01 (0.002 μg of protamine/ml of formulation). After addition of the glycine or glycine/propylene glycol auxiliary substance solution, brief opalescence, which disappeared again after metering in further auxiliary substance solution, occurred in all the solutions. This brief opalescence could be explained by the IP being passed through. Both those formulations which contain glycine and propylene glycol and those without propylene glycol were clear solution, in which the following pH values were determined:

| | | | | pH |
|---|---|---|---|---|
| | Appearance | pH | Appearance | Gly/PG |
| Molar ratio | glycine | glycine | Gly/PG | formu- |
| hGH:protamine | formulation | formulation | formulation | lation |
| 1:0.5 | clear solution | 3.92 | clear solution | 4.40 |
| 1:0.2 | clear solution | 3.91 | clear solution | 4.42 |
| 1:0.1 | clear solution | 3.93 | clear solution | 4.39 |
| 1:0.05 | clear solution | 3.93 | clear solution | 4.38 |
| 1:0.02 | clear solution | 3.97 | clear solution | 4.36 |
| 1:0.01 | clear solution | 3.90 | clear solution | 4.50 |

### Experiment 12

### PH of the Formulations

In both formulations (glycine/propylene glycol and glycine) it was possible to establish a pH of 5.8 without opalescence being observed only with the molar ratios of 1:0.02 and 1:0.01. In all the other hGH/protamine formulations tested, opalescence occurred from a pH of 5.8. After correction of the pH to a value of 6.2, all the solutions which contain glycine and propylene glycol were opalescent, whereas those with glycine remained clear at the low dilutions (molar ratios of 1:0.05; 1:0.02; and 1:0.01).

### Experiment 13

Investigations were carried out with the mannitol formulation for comparison. The preparation process was retained, the experiment was doubled in size and a pH of 7 was established with 0.5 M sodium hydroxide solution before metering in the aqueous protamine solution. This should prevent the mixture from passing through the IP when rendered alkaline later.

The pH measurements of the mixtures of bulk solution, water and auxiliary substance solution gave:

| pH | pH |
|---|---|
| formulation (Gly/PG) | formulation (mannitol) |
| 4.05 | 6.99 |
| 4.20 | 6.18 |
| 4.05 | 6.22 |

### Experiment 13

### PH of the Mixture of Bulk Solution, Water and Auxiliary Substance Solution

Thereafter, the aqueous protamine solution was added and the mixture was topped up to the final weight with water. The pH meter determined the following values for the final formulations:

| | | | | pH |
|---|---|---|---|---|
| | Appearance | pH | Appearance | mannitol |
| Molar ratio | Gly/PG | Gly/PG | mannitol | formu- |
| hGH:protamine | formulation | formulation | formulation | lation |
| 1:0.05 | clear solution | 7.15 | slightly | 7.15 |
| | | | opalescent | |
| 1:0.02 | clear solution | 7.01 | slightly | 7.11 |
| | | | opalescent | |
| 1:0.01 | clear solution | 7.41 | clear | 7.14 |
| | | | solution | |

### Experiment 13

### PH of the Formulation

After testing the pH, a target pH of 6.2 was established with phosphoric acid. The glycine/propylene glycol batch was dissolved to a clear solution, and the mannitol batch was opalescent.

After correction of the pH and transfer into 2R vials, the batches were stored with the following values:

| | Appearance | pH | Appearance | pH |
|---|---|---|---|---|
| Molar ratio | Gly/PG | Gly/PG | mannitol | mannitol |
| hGH:protamine | formulation | formulation | formulation | formulation |
| 1:0.05 | slightly | 6.33 | slightly | 6.12 |
| | opalescent | | opalescent | |
| 1:0.02 | slightly | 6.5 | slightly | 6.4 |
| | opalescent | | opalescent | |
| 1:0.01 | clear | 6.42 | clear | 6.13 |
| | solution | | solution | |

### Experiment 13

### PH of the Final Formulation after Correction of the pH

It is assumed that at a molar ratio of 1:0.01 it is not the influence of certain auxiliary substances but the very low protamine concentration which is the reason for the clarity of the solutions. This experiment has shown that lowering the pH of the final formulations to below a value of 6.5 leads to opalescence in some of the preparations, and this depends on the amount of protamine.

### Experiment 14

In this experiment, correction of the pH during the preparation process was omitted. Only the pH of the final formulations was determined and adjusted slightly. Only a mannitol auxiliary substance solution was employed here, and the hGH and protamine were brought together in molar ratios of 1:0.05 (1 μg of protamine/ml of formulation); 1:0.02 (0.4 μg of protamine/ml of formulation) and 1:0.01 (0.2 μg of protamine/ml of formulation).

| Molar ratio | Appearance | pH |
|---|---|---|
| hGH:protamine | mannitol formulation | mannitol formulation |
| 1:0.05 | slightly opalescent | 6.18 |
| 1:0.02 | slightly opalescent | 6.17 |
| 1:001 | very slightly opalescent | 6.25 |

### Experiment 14

### PH of the Formulation

The pH and the appearance of those formulations which were stored for stability tests after correction of the pH with 1 M phosphoric acid can be seen from the following table:

| Molar ratio | Appearance | pH |
|---|---|---|
| hGH:protamine | mannitol formulation | mannitol formulation |
| 1:0.05 | slightly opalescent | 6.18 |
| 1:0.02 | slightly opalescent | 6.10 |
| 1:0.01 | very slightly opalescent | 6.08 |

### Experiment 14

### PH of the Final Formulation after Correction of the pH

Each of the dilutions is opalescent, and it was not possible to lower the pH below a value of 6 without opalescence being visible.

### Experiment 15

In this experiment, the protamine concentration was reduced considerably compared with earlier experiments. hGH and protamine were employed in molar ratios of 1:0.005 (0.1 μg of protamine/ml of formulation) and 1:0.001 (0.02 μg of protamine/ml of formulation). Both the mannitol and the glycine/propylene glycol recipe were used. The pH of the mixtures of bulk solution, water and auxiliary substance solution was again determined, and the following values resulted:

| Molar ratio | pH | pH |
|---|---|---|
| hGH:protamine | mannitol formulation: | Gly/PG formulation |
| 1:0.005 | 6.09 | 3.57 |
| 1:0.001 | 6.00 | 3.68 |

### Experiment 15

### PH of the Mixture of Bulk Solution, Water and Auxiliary Substance Solution

After correction of the pH to 7, addition of the protamine solution and topping up to the target volume with water, it was possible to determine the following values for the final formulations.

| | Appearance | pH | Appearance | pH |
|---|---|---|---|---|
| Molar ratio | Gly/PG | Gly/PG | mannitol | mannitol |
| hGH:protamine | formulation | formulation | formulation | formulation |
| 1:0.005 | clear | 7.05 | clear | 7.20 |
| | solution | | solution | |
| 1:0.001 | clear | 7.02 | clear | 7.06 |
| | solution | | solution | |

### Experiment 15

### PH of the Formulation

None of the dilutions showed opalescence. After correction of the pH, the following values resulted for the final formulations, all the solutions being clear.

| | Appearance | pH | Appearance | pH |
|---|---|---|---|---|
| Molar ratio | Gly/PG | Gly/PG | mannitol | mannitol |
| hGH:protamine | formulation | formulation | formulation | formulation |
| 1:0.006 | clear | 5.1 | clear | 5.9 |
| | solution | | solution | |
| 1:0.001 | clear | 5.7 | clear | 5.8 |
| | solution | | solution | |

### Experiment 15

### PH of the Final Formulation after Correction of the pH

In this experiment, the fact that in spite of a final pH of 5 no opalescence has occurred is probably to be attributed to the low protamine concentration.

Preparation process of Experiments 16, 17, 18, 19
correction of the pH of the auxiliary substance solution to a required value
introduction of protamine as an additional component of the auxiliary substance solutions; protamine is dissolved directly in the auxiliary substance solution
dilution of the stock auxiliary substance solution with further auxiliary substance solution in order to achieve the desired protamine concentration
cautious metering in of the bulk solution
adjustment of the target pH of the final formulation
centrifuging off of any precipitates which occur
transfer into 2R vials and storage at room temperature or at 2-8° C.

### Experiment 16

The preparation process was modified inasmuch as protamine was not added to the auxiliary substance as an aqueous solution but was dissolved directly in the auxiliary substance solution, which was adjusted to a pH of 7. The bulk solution was metered into the auxiliary substance solution, which now contains the protamine (75 μg of protamine/ml of formulation) in a high excess, in 30 μl steps with constant shaking.

The batches showed no opalescence, and the following values were determined by means of a pH meter.

| | Appearance | pH | Appearance | pH |
|---|---|---|---|---|
| Molar ratio | Gly/PG | Gly/PG | mannitol | mannitol |
| hGH:protamine | formulation | formulation | formulation | Formulation |
| 1:267 | clear | 12.09 | clear | 10.96 |
| | solution | | solution | |

### Experiment 16

### PH of the Formulation

Thereafter, the pH was lowered to a target pH of 6.0 with 1 M phosphoric acid, and the formulations remained clear.

### Experiment 17

The preparation in the experiment was carried out analogously to Experiment 16, with the difference that in this experiment the auxiliary substance solution had a pH of 3.6 instead of 7.

| | Appearance | pH | Appearance | pH |
|---|---|---|---|---|
| Molar ratio | Gly/PG | Gly/PG | mannitol | mannitol |
| hGH:protamine | formulation | formulation | formulation | formulation |
| 1:267 | clear | 12.70 | clear | 12.60 |
| | solution | | solution | |

### Experiment 17

### PH of the Formulation

The pH was again corrected to 6 with 1 M phosphoric acid, opalescence being observed in none of the formulations.

Experiments 16 and 17 showed that with an excess of protamine in a molar ratio of 1:267 (75 μg of protamine/ml of formulation), no opalescence occurs in the formulations, regardless of the preset pH and the composition of the auxiliary substance solution.

### Experiments 18, 19

hGH and protamine were employed for both recipes in the molar ratios of 1:100 (53.5 μg of protamine/ml of formulation); 1:10 (5.35 μg of protamine/ml of formulation); 1:1 (0.53 μg of protamine/ml of formulation); 1:0.1 (0.05 μg of protamine/ml of formulation). The four final formulations were finally corrected to the pH of 6.5; 6.2; 6.0 and 5.8. In the auxiliary substance solutions, the pH was adjusted to 7 after the addition of protamine.

Before this correction, the following values were measured:

| | pH | |
|---|---|---|
| | glycine | pH |
| Molar ratio | auxiliary | mannitol auxiliary |
| hGH:protamine | substance solution | substance solution |
| 1:100 | 12.44 | 12.69 |
| 1:10 | 9.23 | 11.25 |
| 1:1 | 7.64 | 6.87 |
| 1:0.1 | 6.56 | 6.38 |

### Experiments 18, 19

### PH of the Auxiliary Substance Solutions

In the solutions measured, a pH of 7 was subsequently established.

After addition of the bulk solution, the following values were determined:

| | Appearance | pH | Appearance | |
|---|---|---|---|---|
| Molar ratio | glycine | glycine | mannitol | pH |
| hGH:protamine | formulation | formulation | formulation | mannitol formulation |
| 1:100 | clear solution | 7.04 | clear solution | 7.05 |
| 1:10 | slightly opalescent | 7.14 | slightly opalescent | 7.12 |
| 1:1 | intense opalescence | 7.12 | intense opalescence | 7.11 |
| 1:0.1 | slight opalescence | 7.00 | slight opalescence | 7.05 |

### Experiments 18, 19

### PH of the Formulation

The pH correction of Experiments 18 and 19 was carried out with 1 M phosphoric acid or 0.1 M phosphoric acid.

The appearance of the solutions is summarized in the following table.

| | | pH | | |
|---|---|---|---|---|
| Molar ratio | Appearance | glycine | Appearance | pH |
| hGH:protamine | glycine formulation | formulation | mannitol formulation | mannitol formulation |
| 1:100 | clear solution | 6.5 | clear solution | 6.5 |
| 1:100 | clear solution | 6.2 | clear solution | 6.2 |
| 1:100 | clear solution | 6.0 | clear solution | 6.0 |
| 1:100 | clear solution | 5.8 | clear solution | 5.8 |
| 1:10 | slightly opalescent | 6.5 | slightly opalescent | 6.5 |
| 1:10 | slightly opalescent | 6.2 | slightly opalescent | 6.2 |
| 1:10 | slightly opalescent | 6.0 | slightly opalescent | 6.0 |
| 1:10 | slightly opalescent | 5.8 | slightly opalescent | 5.8 |
| 1:1 | slight opalescence | 6.5 | intense opalescence | 6.5 |
| 1:1 | intense opalescence | 6.2 | intense opalescence | 6.2 |
| 1:1 | intense opalescence | 6.0 | intense opalescence | 6.0 |
| 1:1 | intense opalescence | 5.8 | intense opalescence | 5.8 |
| 1:0.11 | slight opalescence | 6.5 | slight opalescence | 6.5 |
| 1:0.1 | slight opalescence | 6.2 | slight opalescence | 6.2 |
| 1:0.1 | slight opalescence | 6.0 | slight opalescence | 6.0 |
| 1:0.1 | intense opalescence | 5.8 | slight opalescence | 5.8 |

### Experiments 18, 19

### Appearance and pH of the Adjusted Final Formulations

The results of Experiments 18 and 19 illustrate clearly that only at a molar hGH/protamine ratio of greater than 1:10 is it possible to obtain clear solutions. However, the choice of auxiliary substance solution has little influence here, since the same results were achieved for the mannitol and the glycine formulation.

In further experiments, 1:13 was determined as the minimum molar ratio of somatropin to protamine.

The features of the invention which have been disclosed in the preceding description, the claims and the drawings can be essential both individually and in any desired combination for realizing the invention in its various embodiments.

1-17. (canceled)

18. Storage-stable liquid formulation comprising an active substance and protamine, wherein the liquid formulation differs from a suspension and the active substance has a molecular weight of from about 10,000 to about 100,000 Da or is a peptide which comprises more than 50 amino acids.

19. Liquid formulation according to claim 18, characterized in that the liquid formulation is a clear liquid formulation.

20. Liquid formulation according to claim 18, characterized in that the liquid formulation has an opalescence which is not more intense than the opalescence of reference solution IV as defined in Ph.Eu. 1997, 2.2.1.

21. Liquid formulation according to claim 18, characterized in that the protamine is salmon sperm protamine.

22. Liquid formulation according to claim 18, characterized in that the molar ratio of active substance to protamine is at least 1:2 or more, preferably at least 1:10 and more preferably at least 1:100.

23. Liquid formulation according to claim 18, characterized in that the molar ratio of active substance to protamine is at least 1:13.

24. Liquid formulation according to claim 18, characterized in that the active substance is chosen from the group comprising peptides, polypeptides and proteins.

25. Liquid formulation according to claim 18, characterized in that the active substance is chosen from the group which comprises growth factors, cytokines, interferons, interleukins and antibodies.

26. Liquid formulation according to claim 18, characterized in that the active substance is hGH.

27. Liquid formulation according to claim 18, characterized in that the liquid formulation has a pH of from about 4 to 10, preferably from about 5.5 to 7.

28. Liquid formulation according to claim 18, characterized in that the liquid formulation contains at least one further auxiliary substance, the auxiliary substance being chosen from the group comprising glycine, propylene glycol, mannitol and poloxamer 188.

29. Liquid formulation according to claim 18 for use in a method for treatment of a disease.

30. Use of protamine for the preparation of a liquid formulation according to claim 18.

31. Use of protamine as a solubilizing agent in the context of the preparation of a liquid formulation according to claim 18.

32. Process for the preparation of a liquid formulation comprising an active substance and protamine according to claim 18, comprising the steps:
a) provision of protamine;
b) provision of the active substance; and
c) formulation of protamine and the active substance to give a liquid formulation.
